(19) United States
(12) Patent Application Publication
(10) Pub. No.: US 2021/0196121 A1
(43) Pub. Date: Jul. 1, 2021

(54) Patient Monitoring System and Method With Automated Patient Monitor Transfer

(71) Applicant: GE Precision Healthcare LLC, Wauwatosa, WI (US)

(72) Inventors: Lauri Tapio Aarnio, Helsinki (FI); Liisa-Maria Kristiina Jarvela, Helsinki (FI); Stanislav Korkiainen, Helsinki (FI)

(73) Assignee: GE Precision Healthcare LLC, Wauwatosa, WI (US)

(21) Appl. No.: 16/731,745

(22) Filed: Dec. 31, 2019

(51) Int. Cl. A61B 5/00; G16H 40/67; G16H 10/60

(52) U.S. Cl. CPC A61B 5/002; A61B 5/7445; G16H 10/60; G16H 40/67; A61B 5/7475

(57) ABSTRACT

A method of monitoring a patient includes operating a first wireless sensing device to measure at least first physiological parameter from a patient and wirelessly transmit a first parameter data based on the first physiological measurements. The first parameter data is received at a first patient monitor from the wireless sensing devices. First physiological information is then displayed on a first display associated with the first patient monitor, wherein the first physiological information is based on the parameter data. The first wireless sensing device is detected in a predefined area associated with a second patient monitor and identification information of the first wireless sensing devices is then communicated to the second patient monitor. The second patient monitor is then operated to receive the first parameter data from the first wireless sensing device and to display the first physiological information on a second display associated with the second patient monitor.

### BACKGROUND

The present disclosure generally relates to medical devices and, more particularly, to patient monitoring devices for monitoring patient physiology and health status, especially wireless monitoring devices and systems.

Monitoring physiological parameters of a patient is an important part of patient care and physicians often desire to continuously monitor multiple physiological parameters of their patients. Well-known parameters of patient health include blood pressure, oxygen saturation (SpO2), and features of the electrocardiogram (ECG). Thus, patient monitoring often involves the use of several sensing devices simultaneously to perform multiple physiological monitoring modalities, such as a pulse oximeter, a blood pressure monitor, a heart monitor, a temperature monitor, etc. Many patient monitoring devices offer multi-modality patient monitoring, where multiple different sensing devices for sensing different physiological parameters can be connected to a single patient monitor that is configured to collect, process, and/or display physiological information describing the patient's health condition.

Continuous physiological monitoring of the patient causes well-known burdens to the clinical environment. Managing patient monitoring devices occupies significant amounts of clinician time and resources, from connecting the patient up to the monitors to ensuring proper and continuous operation of the monitoring devices. Wireless communication technology leveraged for patient monitoring mitigates some problems associated with patient monitoring management, such as the need for clinicians to manage cable clutter and physical connection and disconnection of the patient to a monitoring device. In addition, wireless instrumentalities greatly reduce the burden associated with cable management, which can hamper patient movement and cause frequent sensor detachment, which disrupts patient monitoring.

However, wireless patient monitoring devices bring their own challenges for providing continuous and accurate patient monitoring. When cables are removed from monitoring devices, new systems and methods must be implemented to ensure that monitoring devices are properly connected to and associated with the correct patient. Existing solutions for associating wireless sensing devices and patient monitors to a monitored patient are exemplified and described at U.S. Pub. 20180177397, which is incorporated herein by reference in its entirety.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a method of monitoring a patient includes operating a first wireless sensing device to measure at least first physiological parameter from a patient and wirelessly transmit a first parameter data based on the first physiological measurements. The first parameter data is received at a first patient monitor from the wireless sensing devices. First physiological information is then displayed on a first display associated with the first patient monitor, wherein the first physiological information is based on the parameter data. The first wireless sensing device is detected in a predefined area associated with a second patient monitor and identification information of the first wireless sensing devices is then communicated to the second patient monitor. The second patient monitor is then operated to receive the first parameter data from the first wireless sensing device and to display the first physiological information on a second display associated with the second patient monitor.

On embodiment of a patient monitoring system includes at least one wireless sensing device configured to measure at least a first physiological parameter from a patient and wirelessly transmit a first parameter data based on the first physiological parameter measurements. A first patient monitor is configured to receive at least the first parameter data and to display first physiological information on a first display based on the first parameter data. A detector is configured to detect the presence of first patient monitor and/or the first wireless sensing device in a predefined area. A second patient monitor is communicatively connected to the detector and configured to receive parameter data from the wireless sensing device so as to provide patient monitoring of a patient located in the predefined area. The second patient monitor is further configured to, following detection of the first patient monitor and/or the first wireless sensing device in the predefined area by the detector, receive at least the first parameter data and display the physiological information on a second display associated with the second patient monitor so as to perform patient monitoring with the second patient monitor.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a schematic diagram of an exemplary embodiment of a patient monitoring system.

FIG. 2 depicts an exemplary embodiment of a patient monitoring system and exemplary method implemented in an operating ward of a healthcare facility containing several operating rooms.

FIGS. 3-5 are flow charts exemplifying methods of wireless patient monitoring according to embodiments of the present disclosure.

### DETAILED DESCRIPTION

The inventors have recognized that improved wireless patient monitoring systems and methods are needed to facilitate transfer of patient monitoring responsibility from one patient monitor to another, such as when a patient is moved from one location to another in healthcare facility. Different wards and functions in healthcare facilities have different standard monitoring devices installed, such as to provide monitoring functionality for the specific healthcare function provided in that ward or area of that healthcare facility. One such example is in an operating room (OR) where an OR patient monitor is configured to monitor physiological parameters of the patient needed for patient assessment during surgery and for purpose of maintaining anesthesia, and the OR patient monitor is configured to display the physiological information of the patient in particular ways configured for monitoring the patient during anesthesia and surgery. Similarly, specific patient monitor systems are installed at other locations in the healthcare facility, including intensive care units (ICUs), physical therapy units, labor and delivery, emergency care unit, bedside monitors in patient rooms, etc. Additionally, portable transport monitors may be provided which are configured to monitor the patient in transit from one location to another, such as from the patient room to the OR, or from the OR to the ICU.

In a healthcare environment, such a hospital, the patient monitors are typically configured to communicate patient monitoring data, such as measured parameter data and physiological information derived from the parameter data, on a host network of the medical facility for storage in the patient's medical record. The host network infrastructure allows the patient monitoring data to be accessible at any location that has computer access to the host network, and thus can be reviewed, for example, at a nurse's station or even remotely by logging into the host network. Additionally, the host network allows multiple different patient monitors to be used at different times to monitor the patient, wherein all such data can be aggregated into the patients' medical record and accessible by any subsequent patient monitor that connects to the wireless sensing devices and is configured to monitor the patient's physiology.

Patient monitoring is often already started in one care area prior to transporting the patient from one care area to another, where wireless sensing devices are attached to the patient and are communicating to a patient monitor fixed at a particular location. In order to provide continuous monitoring during patient transport, a portable transport monitor may be used and connect to receive physiological parameter data transmitted by the wireless sensing devices. In various embodiments, the portable transport monitor may be connected to the host network of the healthcare facility and may transmit the parameter data and or other physiological information to the host network during the monitoring period. Alternatively or additionally, the portable transport monitor may store the parameter data and display physiological information based thereon during the patient transport so that clinicians can continue to monitor the patient condition. To provide just one example, in a case where a patient being treated in an emergency room needs emergency surgery, a portable transport monitor may be utilized. In such a scenario, monitoring may begin using a patient monitor and emergency room. Continuous monitoring during transport may be facilitated by switching patient monitoring to a portable transport monitor, which can be utilized until such time as the patient arrives at the OR and monitoring can be switched over to an OR patient monitor.

The same wireless physiological sensors may be utilized with all of the described types of monitors found in a typical healthcare facility. Such wireless sensing devices need to be associated with each monitor to which they are communicating. The association between the wireless sensing devices and the patient monitor allows for effective and secure patient monitoring, where the patient monitoring receiving the parameter data from the wireless sensors has established a secure communication link and has awareness of the patient to which the wireless sensing devices are attached. Thus, association is more than just a typical radio communication between two devices and instead requires exchange of identification information between the devices so that the patient monitor can link the parameter data and other physiological information to the patient, such as for storage in the patient's medical record, and so that parameter data of different patients is not accidentally confused.

The inventors have recognized that the transfer of the wireless sensing devices from one patient monitor to another is a time consuming and high risk task in the clinician workflow. Performance of this handoff between patient monitors occupies clinician time and attention and thus can detract from patient care. Moreover, inaccuracy of the transfer can lead to inaccurate or insufficient patient monitoring and/or occupy additional clinician time in order to fix any mistakes made in the transfer. Typically, the transfer of patient monitoring, and thus receipt of the physiological parameter data from the wireless sensing devices, is performed manually by clinicians and requires entering patient information and for the clinician to manage pairing the patient monitor to each of the wireless sensing devices. Furthermore, to the extent that such transfer is performed offline and the patient monitor is not contemporaneously connected to the host network, the patient monitoring may initially be performed without historical parameter data describing the history of the patient's physiological parameter, such as describing the previous minutes, hours, or days of the monitored physiological parameter. Thus, monitoring may be initially inaccurate or incomplete upon transfer. Moreover, manual entry of the patient information can lead to inaccurate pairing resulting from human error, such as errors in the patient information (e.g., name, date of birth, height, weight, patient ID, etc.) or errors in how and which sensors get paired with the monitor.

In view of the foregoing challenges and problems recognized by the inventors as a result of their extensive experimentation and research in the relevant field, the disclosed system and method have been developed to automate transfer of wireless patient monitoring by wireless sensing devices from one patient monitor to another. The disclosed automated transfer may be an automatic transfer of monitoring responsibilities from one patient monitor to another, or may be an automatic transfer of identification information of the wireless sensing devices to a new patient monitor, and an automated presentation of an approval request to a clinician requesting clinician input to approve receipt of data from the wireless sensing devices at the new patient monitor.

In one embodiment, a detector is provided that detects the presence of a first patient monitor and/or one or more wireless sensing devices in a pre-defined area so as to provide patient monitoring of a patient located in that area. For example, where the first patient monitor is a portable transport monitor configured to monitor the patient in transit to an operating room (OR), and the second patient monitor is an OR patient monitor configured to monitor a patient in an OR. A detector may be configured to detect the presence of the first patient monitor and/or the wireless sensing devices associated therewith once the patient enters the OR. The system then transfers all associations and patient information, such as patient ID, relevant health information (e.g., pacemaker information, etc.) from the portable transport monitor to the OR patient monitor automatically. For example, all wireless sensing devices and the monitoring configuration may be automatically detected and transferred to the OR patient monitor with minimal or no user interaction, save possibly in an approval input of the transfer from a clinician.

Similarly, when a patient is leaving the OR, or another predefined area, the system may be configured to transfer the patient monitoring back to the transport monitor as the patient leaves the OR, and that transfer can be provided in the same automated way. Alternatively or additionally, the system may be configured to detect that the sensors should be transferred to another device, such as by detecting that the patient has arrived in the predefined area or is leaving the predefined area, but no such patient monitoring device is available. For example, if the patient is being moved out of the OR without the transport monitor or the transport monitor has run out of battery or malfunctioned. The system may be configured to raise an alarm to indicate that the association of the wireless sensing devices to a patient monitor is about to be disrupted and that a new patient is not available to continue receiving the physiological parameter data from the wireless sensing devices, and thus to continue the patient monitoring.

Accordingly, the patient monitoring system and method described herein greatly improves patient safety and care quality by reducing the burden of patient monitoring on clinicians and reducing the incidents of human error in the monitoring workflow. The disclosed system and method providing automatic transfer of wireless patient monitoring associations from one patient monitor to the next makes it possible to provide continued patient monitoring during patient transport from one care location to another with minimal or no clinician intervention.

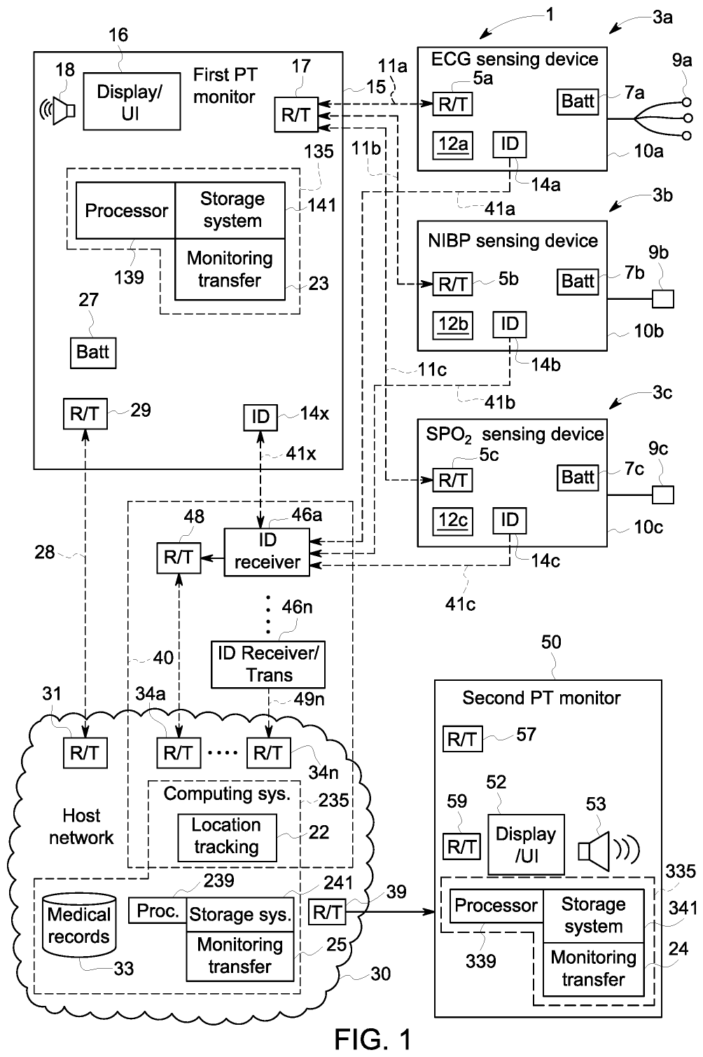

FIG. 1 exemplifies a patient monitoring system one that includes one or more wireless sensing devices (e.g., 3a-3c) each measuring different physiological parameters from a patient. These sensing devices 3a-3c may be associated with any one or more patient monitors in the system 1. In the scenario depicted at FIG. 1, the sensing devices 3a-3c are associated with and communication physiological parameter data to a first patient monitor 15. The first patient monitor 15 may be configured to communicate with a computer network for the medical care facility, i.e. host network 30. The host network 30 contains medical records database 33 and may further be configured to facilitate patient monitoring such as to provide location tracking information from a location tracking system, as described herein. The system 1 includes at least one additional patient monitor, and may include multiple patient monitors representing the different types of patient monitors that may be found in a healthcare facility.

In the depicted example, the system 1 includes first patient monitor 15, and second patient monitor 50. Each patient monitor 15, 50 is configured to receive parameter data from each of the wireless sensing devices 3a-3c and to display physiological information based on the parameter data on respective display 16, 52 associated with the patient monitor 15, 50. The display 16, 52 may comprise part of a user interface configured to receive input, such as clinician input approving receipt of monitoring transfer. For example, each display 16, 52 may be a touch screen display, for example, controllable to present the transfer request and receive clinician approval input. The displayed physiological information may take any format, many of which are well known in the art, including graphical waveforms, values, trends, patient health indicators, or the like. In various embodiments the physiological information displayed by the patient monitor 15, 50 may be based on parameters data recorded by the one or more wireless sensing devices 3a-3c overtime. The patient monitors 15, 50 may further be configured to assess patient data for purposes of monitoring for alarm conditions and to generate alarm notices or alerts, such as visual alerts on the respective display 16, 52 and/or auditory alarms via the respective speaker 18, 53.

The first patient monitor 15 and the second patient monitor 50 may be different types of patient monitors serving different monitoring functions at different times in the continuous monitoring of the patient. For example, the first patient monitor may be a portable transport monitor configured to monitor the patient in transit. Thus, for example, the first patient monitor 15 may be battery powered and thus contain a battery 27. The second patient monitor 50 may be, for example, a stationary monitor installed at a patient care location. To provide one example, the second patient monitor 50 may be an OR patient monitor configured to monitor a patient while they are located in an operating room. Accordingly, the second patient monitor 50 may be configured to provide patient monitoring features geared toward the specific monitoring environment, such as an OR patient monitor configured to monitor a patient during anesthesia and surgery. Similarly, the first patient monitor 15 may be configured for a particular monitoring environment such as to provide basic physiological information about the patient during patient transport.

Each wireless sensing device 3a-3c includes one or more sensors 9a-9c for measuring physiological parameter data from a patient, and also includes a data acquisition device 10a-10c that receives the physiological parameter measurements from the sensors 9a-9c and transmits a parameter dataset based on those measurements to the patient monitor 15, 50 via communication link 11a-11c. The sensors 9a-9c may be connected to the respective data acquisition device 10a-10c by wired or wireless means. The sensors 9a-9c may be any sensors, leads, or other devices available in the art for sensing or detecting physiological information from a patient, which may include but are not limited to electrodes, lead wires, or available physiological measurement devices such as pressure sensors, flow sensors, temperature sensors, blood pressure cuffs, pulse oximetry sensors, or the like. In the depicted embodiment, a first sensing device 3a is an ECG sensing device having sensors 9a that are ECG electrodes. A second sensing device 3b is a non-invasive blood pressure (NIBP) sensing device with a sensor 9b that is a blood pressure cuff including pressure sensors. A third sensing devices 3c is a peripheral oxygen saturation (SpO2) monitor having sensor 9c that is a pulse oximetry sensor, such as a standard pulse oximetry sensor configured for placement on a patient's fingertip. It should be understood that the patient monitoring system 1 of the present disclosure is not limited to the examples of sensing devices provided, but may be configured and employed to sense and monitor any physiological parameter of the patient. The examples provided herein are for the purposes of demonstrating the invention and should not be considered limiting.

The data acquisition device 10a-10c of each exemplary sensing devices 3a-3c may include an analog-to-digital (A/D) converter, which may be any device or logic set capable of digitizing analog physiological signals recorded by the associated sensor 9a-9c. For example, the A/D converter may be Analog Front End (AFE) devices. Each data acquisition device 10a-10c may further include a processing unit 12a-12c that receives the digital physiological data from the A/D converter and creates physiological parameter data for transmission to the patient monitor 15 and/or to the host network 30. Each data acquisition device 10a-10c may be configured differently depending on the type and function of sensing devices, and may be configured to perform various signal processing functions and/or sensor control functions. To provide just a few examples, the processing unit 12a in the ECG sensing device 3a may be configured to filter the digital signal from the ECG sensors 9a to remove artifact and/or to perform various calculations and determinations based on the recorded cardiac data, such as heart rate, QRS interval, ST segment/interval, or the like. The processing unit 12b in the NIBP monitor 3b may be configured, for example, to process the physiological data recorded by the sensors 9b in a blood pressure cuff to calculate systolic, diastolic, and mean blood pressure values for the patient. The processing unit 12c of the SpO2 sensing device 3c may be configured to determine a blood oxygenation value for the patient based on the digitized signal received from the pulse oximetry sensor 9c.

Accordingly, each processing unit 12a-12c may develop physiologic parameter data that, in addition to the recorded physiological data, also includes values measured and/or calculated from the recorded physiological data. The respective processing units 12a-12c may then control a receiver/transmitter 5a-5c in the relevant sensing devices 3a-3c to transmit the physiological parameter data via communication link 11a-11c. The physiological parameter data transmitted from the respective sensing devices 3a-3c may include the raw digitized physiological data, filtered digitized physiological data, and/or processed data indicating information about the respective physiological parameter measured from the patient. Additionally, one or more of the data acquisition devices 10a-10c may be configured to compare the physiological parameter data to one or more alarm thresholds to determine the presence of an alarm condition—i.e., detect an alarm event based on the physiological parameter data from one or more of the wireless sensing devices 3a-3c.

Upon detection of an alarm event by the respective sensing device 3a-3c, an alarm may be generated either by the sensing device or the associated patient monitor 15, 55 (e.g. via speaker 18, 53 and/or display 16, 52). Alternatively, the patient monitor 15, 50 may be configured to assess the physiological data and to detect the alarm event. Notice of the alarm may be transmitted from the patient monitor 15, 50 to host network 30. In certain embodiments, nursing stations or other central clinician station may be connected to the host network 30 and configured to further display patient monitoring and/or alarm information.

The sensing devices 3a-3c may be further configured to transmit the parameter data along with identification information identifying the respective sensing device 3a-3c to the receiving patient monitor 15, 50, for example. The identification information may include an identification number associated with the respective individual sensing device 3a-3c, which may follow a standard protocol and may indicate the type of sensing device, in addition to the unique identifier for the particular instance of that sensing device 3a-3c. In certain embodiments, the sensing device 3a-3c may have an identification transmitter 14a-14c that communicates with a location tracking system 40, as described herein below.

The alarm events may be triggered by analysis of the physiological parameter data, such as if alarm limits for the respective parameter data are exceeded (e.g., heart rate low), a parameter message alarm (e.g., apnea), or one or more particular data patterns are detected (e.g., indicating an arrhythmia such as tachy or asystole). Additionally, other alarm types may be generated, such as a technical alarm type or an alarm generated regarding treatment delivery to the patient. A technical alarm type is generated based on and/or as a result of a function of the sensing device 3a-3c, the patient monitor 15, 50 or the like, and/or some component thereof. Examples of technical alarm types are low battery alerts (e.g., batteries 7a-7c in the wireless sensing devices 3a-3c), sensor off alerts (e.g., sensor(s) 9a-9c are not properly connected to the patient), sensor malfunction alerts (e.g., sensor(s) 9a-9c is not functioning properly), device malfunction alerts (e.g., sensing device 3a-3c is not functioning properly), data transmission malfunction alert (e.g., there is a problem with one or more communication links 11a-11c, 28, 38), or a technical problem regarding the function of a patient monitor 15, 50. For example, a technical alarm may be generated if one or more of the sensing devices 3a-3c exit the predefined area associated with the respective patient monitor 15, 50 without transfer of patient monitoring association and/or function to another patient monitor.

The system 1 includes software for transferring monitoring association and/or primary patient monitoring responsibilities of recording and/or transmitting the patient monitoring data to the host network 30 between patient monitors (e.g. between the first patient monitor 15 and the second patient monitor 50). The patient monitoring software includes monitoring transfer modules (e.g. 23-25) installed on various components within the system 1. Each patient monitor 15, 50 has an instance of the monitoring transfer module 23, 24, which facilitates transfer of patient monitoring responsibilities to and from the respective patient monitor 15, 50. For example, the monitoring transfer module 23, 24 may be configured to receive and assess identification information of the respective wireless sensing devices 3a-3c and to execute steps for receiving parameter data therefrom. For example, the monitoring transferring modules 23, 24 may be configured to interface with the location tracking system 40 and or a detector configured to detect the presence of one or more wireless sensing devices 3a-3c in a predefined area associated with the respective patient monitor 15, 50. Alternatively or additionally, the monitoring transfer module 23, 24 of each patient monitor 15, 50 may be configured to display a transfer request, such as on the display 16, 52 of the respective patient monitor, and to receive a clinician input approving receipt of parameter data and/or other patient monitoring information from the sensing devices 3a-3c on the patient. The monitoring transfer software 23, 24 may further be configured for communication with the host network 30 in order to facilitate patient monitoring functionality, such as to receive patient identification information for a patient, historical first parameter data for the patient, and/or patient medical information for the patient via the host network 30. Additionally, in some embodiments, the monitoring transfer software 23, 25 on each patient monitor 15, 50 may be configured to facilitate direct communication between the patient monitors 15, 50, such as wireless communication via Wi-Fi, Bluetooth, nearfield communication (NFC), MBAN, or other protocol.

The host network 30 may also store and implement software for facilitating monitoring transfer, represented as monitoring transferring module 25. The monitoring transfer software 25 on the host network 30 may be configured to track monitoring connectivity of each patient monitor 15, 50 and/or to communicate monitoring responsibility designations for associations between a patient monitor 15, 50 and one or more sensing devices 3a-3c on a patient. Additionally, the monitoring transfer software 25 may facilitate the retrieval of patient medical information and/or historical parameter data for the patient upon transfer of monitoring association to a new patient monitor 15, 50.

As will be understood by a person having ordinary skill in the art in view of the present disclosure, the monitoring transfer software 23-25 includes executable instructions stored in a storage system 141, 241, 341 and executable by a processor 139, 239, 339 of a respective computing system 135, 235, 335. The processing systems 139, 239, 339 each includes one or more processors, which may be a microprocessor, a general purpose central processing unit, an application-specific processor, a microcontroller, or any type of logic-based device and may also include circuitry that retrieves and executes software from storage system 141, 241, 341. Each storage system 141, 241, 341 can comprise any storage media, or group of storage media, readable by the respective processing system 139, 239, 339 and capable of storing software. The storage system 141, 241, 341 can include volatile and non-volatile, removable and non-removable media implemented in any method or technology for storage of information, such as computer-readable instructions, data structures, program modules, or other data.

The various devices in the wireless patient monitoring system 1 are configured for wireless communication, and thus each have one or more wireless receivers/transmitters configured for executing one or more wireless communication protocols. In the example of FIG. 1, each sensing device 3a-3c has a receiver transmitter 5a-5c configured to communicate via a protocol to establish the communication link 11a-11c with the receiver/transmitter 17 of the patient monitor 15. Likewise, the receiver transmitter 5a-5c are configured to establish a communication link with the receiver/transmitter 57 of the second patient monitor 50. Depending on the communication roles of the various devices on the network, each receiver/transmitter may include separate a receiving and transmitting devices, or may include an integrated device providing both functions, such as a transceiver. Similarly, the patient monitoring devices 15, 50 include receiver transmitters 29, 59 for communicating with respective receiver transmitters 31, 39 of the host network 30.

In various embodiments, the patient monitors may have one receiver transmitter module configured for communicating via different protocols and enabling wireless communication with both the sensing devices 3a-3c and the host network 30. In various embodiments, each of the receiver transmitters 5a-5c, 17, 29, 31, 39, 57, and 59 are configured for communication on a predefined network via certain wireless protocols, which may include one or more of, but are not limited to Bluetooth, Bluetooth low energy (BLE), ANT, ZigBee, Wi-Fi, etc. In certain embodiments, the receiver transmitters 5a-5c and 17, 57 may be by the area network (BAN) devices, such as medical body area network (MBAN) devices, that operate as a wireless network. Communication between the patient monitors 15, 50 and the host network 30 may be by the same or different wireless protocol means.

The system 1 is configured to enable detection of wireless sensing devices and/or patient monitors in predefined areas associated with other patient monitors. For example, the system 1 is configured to automatically detect when sensing devices 3a-3c and/or the portable first patient monitor 15 enter an OR or other predefined area associated with the second patient monitor 50. In one embodiment, present in the predefined area may be detected using radio frequency identification (RFID), such as where each sensing device 3a-3c and/or the patient monitor 15 have RFID tags detectable by an RFID receiver. With reference to FIG. 1 the sensing devices 3a-3c may each incorporate an identification tag 14a-14c, which may be an active or passive RFID tag depending on the system configuration. Similarly, the first patient monitor 15, in embodiments where the first patient monitor 15 is a portable device, may also include an identification transmitter 14x configured similarly. Multiple identification receivers 46a-46n are positioned around the healthcare facility and are configured to detect the identification transmitters 14 in the vicinity of the respective detector 46a-46n.

In certain embodiments, the identification transmitters 14, and identification receivers 46 may be part of a location tracking system 40 of networked devices communicatively connected via the host network 30. In other embodiments, each identification receiver 46a-46n may be associated with and communicatively connected to a patient monitor at a fixed location, such as the second patient monitor 50. In such an embodiment, the ID receiver 46, or detector, communicates identification information received from one or more identification transmitters 14 in order to facilitate association between the connected patient monitor, such as the second patient monitor 50, and respective sensing devices 3a-3c and/or portable patient monitors 15.

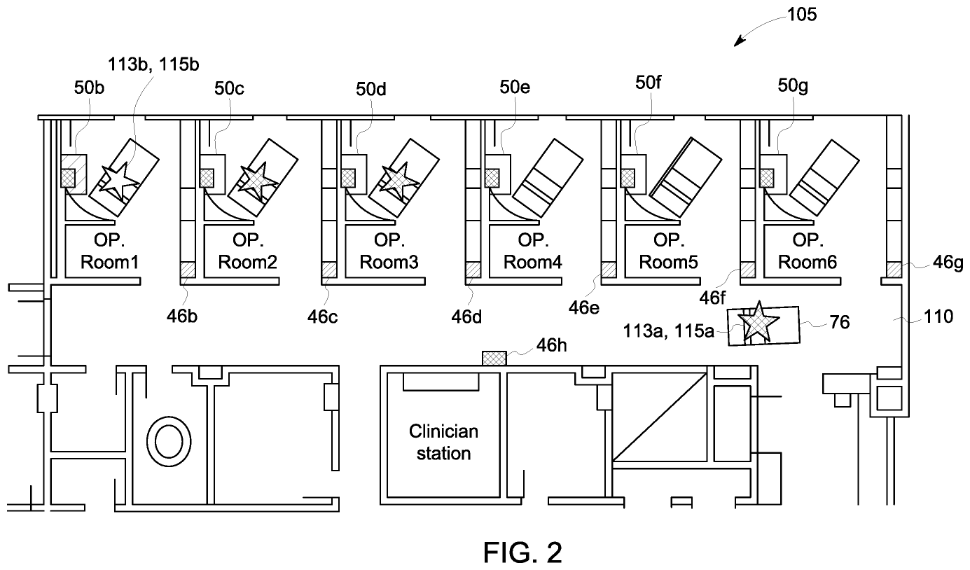

FIG. 2 depicts an exemplary operating ward of a healthcare facility and demonstrates one embodiment and features of the disclosed system 1. Six operating rooms, including OR1 through OR6, are each equipped with an OR patient monitor 50b-50g, respectively. A detector 46 is positioned in each OR and configured to detect the presence of one or more wireless sensing devices 113 and/or patient monitors 115 upon entering that OR. For example, each OR1-OR6 may have a corresponding detector 46b-46g configured for receiving or otherwise communicating with the identification transmitters 14 of the sensing devices 113 and or portable patient monitor 115 as it enters the door of the OR. For example, each detector, 46b-46g may be positioned at or near the door of each OR1-OR6. Each detector 46b-46g may be communicatively connected to the OR patient monitor 50b-50g in the respective OR1-OR6. Thus, upon receiving identification information transmitted from the sensing devices 113 and/or portable patient monitor 115, the detected identification can be transmitted to the respective OR patient monitor 50b-50g.

In the depicted scenario, a monitored patient 76 is being transported to an OR and is shown in a hallway 110 of an operating ward 105 of a healthcare facility. In the depicted example, a location detector 46h is positioned in the hallway 110 and thus may be configured to detect the location of the monitored patient 76 being transported. One or more wireless sensing devices 113a and a portable transport monitor 115a are associated and configured to conduct patient monitoring during the patient transport process. As described above, one or more of the sensing devices 113a and/or patient monitor 115a may include an identification transmitter 14 configured to communicate with a detector 46 in communication proximity thereto.

As the monitored patient 76 enters an OR, the detector 46b-46g detects the presence of the sensing devices 113 and/or portable patient monitor 115a and communicates the identification information to the OR patient monitor 50b-50g in the respective OR. Steps are then executed to transfer patient monitoring association to that OR patient monitor 50b-50g. For example, in operating room OR1 sensing devices 113b and portable patient monitor 115b are provided on the patient. Association between the sensing devices 113b and the OR patient monitor 50b in OR1 has been made such that the OR patient monitor is receiving parameter data from the one or more sensing devices 113b. In the depicted example, the portable patient monitor 115b has stopped receiving and processing the parameter data from the sensing devices 113b after monitoring association was establish with the OR patient monitor. In certain embodiments, the monitoring transfer software module 23 in the portable transport monitor 115b may be configured to receive confirmation of monitoring association with the OR monitor 50b. For example, a confirmation of association with the OR patient monitor 50b may be communicated from that OR patient monitor 50b, such as via Bluetooth or other radio communication protocol operable for such direct and relative short range communication. Alternatively, confirmation of monitoring transfer may be provided through the host network 30, such as via Wi-Fi or other network connection.

Then, as the patient is transported out of the OR, the active patient monitoring role may be transferred back to the portable transport monitor 115b. For example, if the detector 46b detects that the sensing devices 113b and or the portable patient monitor 115b are approaching the door of the OR, detection that the monitored patient is leaving the OR may trigger communication to the portable transport monitor to resume receipt and process of the parameter data from the sensing devices 113b. Alternatively or additionally, the OR patient monitor 50b may be configured to detect a change in signal strength or other indicator that the sensing devices 113b have moved away from the OR patient monitor 50b. In still other embodiments, the OR patient monitor 50b, portable transport monitor 115b, and or sensing devices 113b may be configured to receive clinician input instructing transfer to or from the portable transport monitor 115b.

In one embodiment, the system 1 includes a location tracking system 40 to track location of portable devices, including the wireless sensing devices 3a-3c and the portable patient monitor 15. The location tracking system 40 may be, for example, a real time locating system (RTLS) that provides immediate or real time tracking of the device locations within the healthcare facility or portion thereof.

In the depicted embodiment, a plurality of identification receivers 46a-46n are placed at known locations throughout a care facility. The identifier transmitted by the respective identification transmitter 14a-14c, 14x is received by one of the identification receivers 46a-46n closest to, or otherwise arranged to receive transmissions from, identification transmitters 14a-14c, 14x at that particular location of the tracked device. Each identification receiver 46a-46n then communicates the identifier 41 along with its own receiver identification, to a location tracking module 22. For example, the identification receiver 46a, 46n may communicate the identifier 41 and its own identification with a host network 30 for the care facility via a respective communication link 49a, 49n. The location tracking module 22 then monitors and determines a device location of each monitored device (e.g., 3a-3c, 15) within the care facility. For example, the location tracking module 22 determines a device location based on which identification receiver 46a-46n receives the identifier for that device ID from one or more of the identification transmitters 14a-14c. For example, the location tracking module 22 may access a map or database of the care facility where each identification receiver 46a-46n is associated with a particular location in the care facility (e.g., FIG. 2, 105). The map associating each identification receiver 46a-46n with a location in the care facility may be, for example, uploaded and stored in the computing system 235 of the host network 30 as part of the system configuration.

The patient identification transmitters 14a-14c, 14x communicate with one of a plurality of identification receivers 46a, 46n via a respective communication link 41a-41c, 41x. The communication link 41a-41c, 41x may be by any of various wireless communication protocols and/or platforms, such as Bluetooth, Bluetooth Low Energy (BLE), ZigBee, Wi-Fi, infrared, ultrasound, or by other wireless communication means. In certain embodiments, it is preferable that the transmission range of the patient identifier be limited so that the patient identification transmitters 14a-14c, 14x are only within communication range of one identification receiver 46a-46n at a time. Thus, it may also be beneficial if the system is configured such that the communication signals and protocols do not pass through walls or other structural barriers so that identification receivers 46a, 46n can be placed in adjacent rooms, such as adjacent hospital rooms, without concern of cross-receiving. Accordingly, infrared may provide a good means for the communication links 41a-41c, 41x in other embodiments where line-of-sight limitations are prohibitive, other relatively short-range protocols may be desirable, such as Bluetooth, Bluetooth Low Energy (BLE), or ZigBee, or the like. Alternatively or additionally, communication between the identification receivers 46a, 46n and the identification transmitters 14 may be via a publish-subscribe messaging pattern, or model.

The identification receiver 46a, 46n may communicate with the host network via a separate receiver/transmitter (e.g., 48) that communicates with a respective receiver/transmitter 34 (e.g., 34a) associated with the host network 30. Alternatively, one or more of the identification receivers 46a-46n may have a transmitter incorporated therein capable of transmitting the patient identifier and its own receiver identifier to a respective receiver/transmitter 34a-34n associated with the host network 30. The identification information is communicated to the host network 30 via a respective communication link 49a-49n, which may be by any wireless or wired means and according to any communication protocol. For example, communication may be via a Wi-Fi network for the care facility, or by a dedicated wireless network for the location tracking system 40. For example, in certain embodiments the location tracking system 40 may employ one or more wireless local area networks (WLANs) situated throughout a care facility. In other embodiments, the devices on the location tracking system 40 may utilize the (WMTS) spectrum. Alternatively or additionally, communication between the identification receivers 46a, 46n and the host network 30 may be via a publish-subscribe messaging pattern, or model. In such an embodiment, the identification receivers 46a, 46n may publish information, and the host network 30 may subscribe to the published “messages” from the identification receivers 46a, 46n, or vice versa. Accordingly, the host network 30 does not need to establish a direct communication link with identification receivers 46a, 46n, and vice versa, and each can continue to operate normally regardless of the other.

In the embodiment depicted in FIG. 1, the identification transmitters 14a-14c, 14x are provided in the sensing devices 3a-3c and/or the patient monitor 15 with the identification receivers 46a-46n provided at fixed and known locations throughout the care facility. A person having ordinary skill in the art will understand in light of this disclosure that, in other embodiments the identification receivers 46a-46n may travel with device (such as provided in the sensing devices 3a-3c and/or the portable monitor 15), and transmitters may be provided at fixed locations throughout the care facility to transmit a location identifier of that fixed location. In such an embodiment, the respective sensing devices 3a-3c would receive the location identifier emitted by a location transmitter and would be equipped to determine its own location based on the location identifier received.

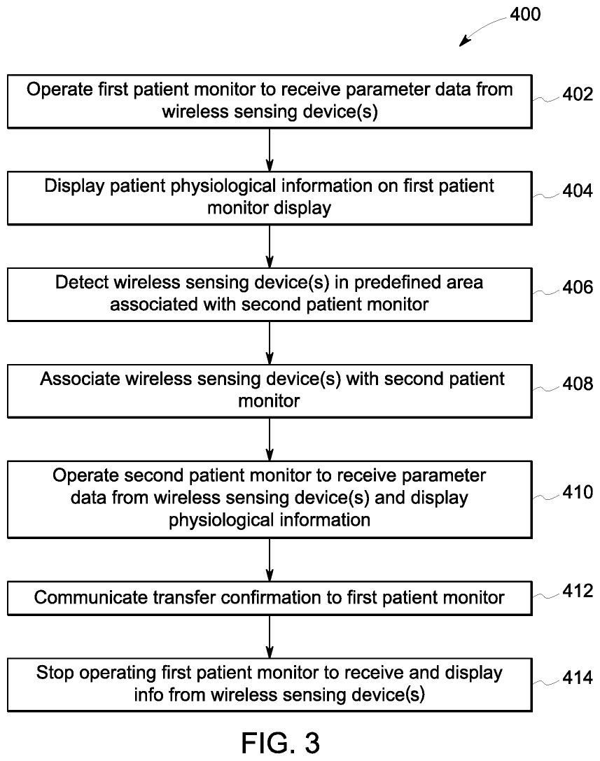

FIGS. 3-5 depict embodiments of methods 400 for monitoring a patient in accordance with the present disclosure. A first patient monitor is operated to receive parameter data from one or more wireless sensing devices at step 402 and physiological information based on that parameter data is displayed on the patient monitor display at step 404, as is routine in patient monitoring. If and when the wireless sensing devices connected to the first patient monitor are detected in a predefined area associated with a second patient monitor, step 406, then the wireless sensing devices will be associated with the second patient monitor at step 408. The second patient monitor is then operated at step 410 to receive the parameter data from the wireless devices and display to physiological information based thereon. A transfer confirmation is then communicated to the first patient monitor at step 412 which then ceases its patient monitoring operations.

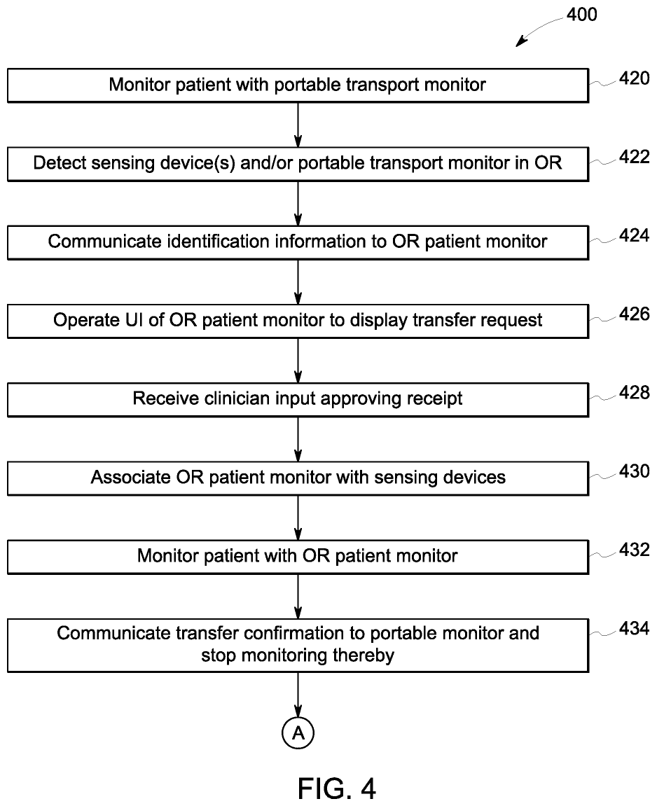

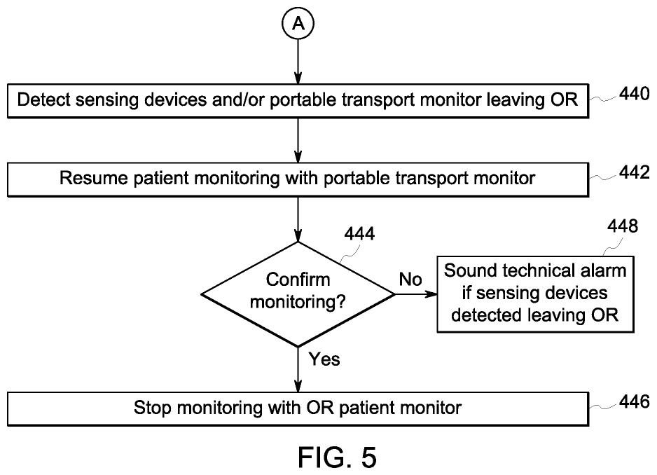

FIGS. 4 and 5 depict another embodiment of a method 400 of monitoring a patient, which is particularly directed towards transferring patient monitoring between a first patient monitor being a portable transport monitor and a second patient monitor being an OR monitor. The patient is monitored using the portable transport monitor while being transported to the OR at step 420. When the wireless sensing devices and/or portable transport monitor are detected in the OR at step 422, such as based on information from the location tracking system 40 or otherwise based on a detector placed in the operating room, the identification information of the sensing devices and/or portable transport monitor are communicated to the OR patient monitor at step 424. The OR patient monitor may be configured to operate the user interface associated with the OR patient monitor to display a transfer request requesting clinician input to approve association with the wireless sensing devices and/or portable transport monitor at step 426. Once the clinician input approving receipt is received at the OR patient monitor at step 428, the OR patient monitor is associated with the sensing devices at step 430 so that the OR patient monitor begins receiving the parameter data from the wireless sensing devices. Thereafter, the patient monitoring is performed by the OR patient monitor at step 432 and a transfer confirmation is sent to the portable patient monitor at step 434. Upon receipt of the transfer confirmation, the portable monitor stop monitoring the patient and, for example, goes into standby mode or shuts down in order to conserve battery. During the patients stay in the operating room, the patient monitoring is performed by the OR patient monitor which, for example, is configured to communicate the patient monitoring information to the host network 30 for storage in the patient's medical record 33.

In various embodiments, the association between the OR patient monitor (e.g. second patient monitor 50) and the wireless sensing devices 3a-3c at step 430 may be a direct communication link, may be through the host network 30, and/or may be via the first patient monitor 15. Thus, for instance, the OR patient monitor may have a receiver transmitter configured to directly receive physiological data transmitted from the wireless sensing devices 3a-3c. In another embodiment, first patient monitor 15 may act as a proxy, such that the links 11a-11c are preserved and the first patient monitor 15 forwards all data to the second patient monitor 50, either indirectly thru the hospital host network 30 infrastructure or directly through wireless transmission means, e.g., Wi-Fi radios. In embodiments where the second patient monitor 50 does not have a wireless interface, then the first patient monitor 15 may wirelessly communicate the physiological data to the host network 30, which may then be forwarded via the host network infrastructure 30 to the second patient monitor 50 (e.g., the OR patient monitor). In such an embodiment where the first monitor as a proxy, the first patient monitor may enter a mode where it appears to be inactive to the user, even if is constantly forwarding data.

At the end of the patient's duration in the OR, steps are executed to transfer the monitoring back to the portable transport monitor. FIG. 5 depicts exemplary steps therefor. Detection that the wireless sensing devices and/or portable transport monitor are leaving the OR is performed at step 420, which may be performed in different ways. For example, a change in signal strength between the wireless sensing devices and the OR patient monitor may be identified and interpreted to indicate that the patient has moved away from the OR monitor or wireless receiver. Alternatively or additionally, detection that the patient is leaving the OR may be based on operation of the transport monitor. For example, the workflow may involve a clinician turning on the portable transport monitor at the end of the surgical procedure and prior to leaving the OR. When the transport monitor turns on it may be configured to automatically resume receipt of transmissions from the wireless sensor devices and other patient monitoring functions.

The portable transport monitor resumes patient monitoring functions, as represented at step 442, including receiving the physiological parameter data transmitted by the wireless sensing devices. Following resumption of the patient monitoring, the portable transport monitor may be configured to communicate a transfer confirmation either directly to the OR patient monitor or indirectly through the host network. Once the monitoring confirmation is received at step 444, such as received at the OR patient monitor or the host network, monitoring functionality by the OR patient monitor is stopped at step 446. If no monitoring confirmation is received confirming monitoring activity by the portable transport monitor, then a technical alarm may be initiated at step 448 to indicate to the clinicians that transfer to the portable transport monitor was not successful and patient monitoring will stop once the patient leaves the OR and thus the OR patient monitor will no longer be able to receive the parameter data from the wireless sensing devices.

This written description uses examples to disclose the invention, including the best mode, and also to enable any person skilled in the art to make and use the invention. Certain terms have been used for brevity, clarity and understanding. No unnecessary limitations are to be inferred therefrom beyond the requirement of the prior art because such terms are used for descriptive purposes only and are intended to be broadly construed. The patentable scope of the invention is defined by the claims, and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

We claim:

1. A method of monitoring a patient, the method comprising:
operating a first wireless sensing device to measure at least a first physiological parameter from a patient and wirelessly transmit a first parameter data based on the first physiological parameter measurements;
receiving the first parameter data from the wireless sensing device at a first patient monitor;
display first physiological information on a first display associated with the first patient monitor, wherein the first physiological information is based on the first parameter data;
detect presence of the first wireless sensing device in a predefined area associated with a second patient monitor;
communicating identification information of the first wireless sensing device to the second patient monitor; and
operating the second patient monitor to receive the first parameter data from the first wireless sensing device and to display the first physiological information on a second display associated with the second patient monitor.

2. The method of claim 1, wherein the first patient monitor is a portable transport monitor configured to monitor the patient in transit and the second patient monitor is an operating room (OR) patient monitor configured to monitor a patient in an OR.

3. The method of claim 2, wherein detecting the presence of the first wireless sensing device in a predefined area includes detecting the first wireless sensing device in the OR.

4. The method of claim 3, further comprising transmitting with an identification transmitter the identification information for the first wireless sensing device, wherein detecting the presence of the first wireless sensing device in the OR includes receiving the identification information at a detector positioned in the OR.

5. The method of claim 4, further comprising:
operating at least a second wireless sensing device to measure at least a second physiological parameter from the patient and to transmit second parameter data;
wherein the identification transmitter further transmits, and the detector further receives, identification information for the second wireless sensing device;
further operating the second patient monitor to receive the second parameter data from the second wireless sensing device and to display the second physiological information on the second display.

6. The method of claim 5, further comprising ceasing operation of the portable transport monitor to receive the first parameter data and the second parameter data after the second patient monitor is receiving the first parameter data and displaying the first physiological information.

7. The method of claim 4, wherein the detector communicates the identification information of the first wireless sensing device to the second patient monitor via a host network.

8. The method of claim 3, further comprising, prior to operating the OR patient monitor to receive the first parameter data:
displaying a transfer request on the second display requesting clinician approval to receive data from the first wireless sensing device; and
receiving a clinician input approving receipt of first parameter data from the first wireless sensing device.

9. The method of claim 1, wherein the first wireless sensing device is configured to transmit the identification information in conjunction with the first parameter data, and wherein detecting the presence of the first wireless sensing device in the predefined area includes receiving the identification information at a detector communicatively connected to the second patient monitor via a host network.

10. The method of claim 9, further comprising retrieving, with the second patient monitor via the host network, patient identification information for the patient, historical first parameter data for the patient, and/or patient medical information for the patient prior to displaying the first physiological information on the second display.

11. The method of claim 1, wherein upon receipt of the identification information of the first wireless sensing device and prior to operating the second patient monitor to receive the first parameter data, further comprising:
displaying a transfer request on the second display requesting clinician approval to receive data from the first wireless sensing device; and
receiving a clinician input approving receipt of first parameter data from the first wireless sensing device.

12. The method of claim 11, further comprising:
operating a plurality of wireless sensing devices to measure a plurality of physiological parameters from the patient;
displaying the transfer request on the second display requesting clinician approval to receive parameter data from all of the plurality of wireless sensing devices for the patient; and
receiving the clinician input approving receipt of parameter data from all of the plurality of wireless sensing devices for the patient.

13. A patient monitoring system comprising:
at least a first wireless sensing device configured to measure at least a first physiological parameter from a patient and wirelessly transmit a first parameter data based on the first physiological parameter measurements;
a first patient monitor configured to receive at least the first parameter data and to display first physiological information on a first display based on the first parameter data;
a detector configured to detect presence of the first patient monitor and/or the first wireless sensing device in a predefined area; and
a second patient monitor communicatively connected to the detector and configured to receive parameter data from wireless sensing devices so as to provide patient monitoring of a patient located in the predefined area;
wherein the second patient monitor is further configured to, following detection of the first patient monitor and/or the first wireless sensing device in the predefined area by the detector, receive at least the first parameter data and to display the first physiological information on a second display associated with the second patient monitor.

14. The system of claim 13, further comprising an identification transmitter configured to transmit identification information that identifies the first patient monitor and/or the first wireless sensing device, wherein the detector detects the presence of the first patient monitor and/or the first wireless sensing device based on the identification information.

15. The system of claim 14, further comprising:
a location tracking system that includes the identification transmitter and the detector and is configured to track the location of the first patient monitor and/or the first wireless sensing device;
wherein the second patient monitor receives the identification information of the first patient monitor and/or the first wireless sensing device from the location tracking system.

16. The system of claim 13, further comprising a host network configured to store patient identification information for the patient, historical first parameter data for the patient, and/or patient medical information for the patient, wherein the detector and the second patient monitor are communicatively connected to the host network;
wherein the second patient monitor is communicatively connected to the host network and is further configured to retrieve the patient identification information, the historical first parameter data, and/or the patient medical information prior to displaying the first physiological information on the second display.

17. The system of claim 13, wherein the first patient monitor is a portable transport monitor configured to monitor the patient in transit and the second patient monitor is an operating room (OR) patient monitor configured to monitor a patient in an OR;
wherein detecting the presence of the first wireless sensing device in a predefined area includes detecting the first wireless sensing device in the OR.

18. The system of claim 17, wherein the system is configured such that the portable transport monitor stops receiving the first parameter data once the OR patient monitor is receiving the first parameter data and displaying the first physiological information.

19. The system of claim 13, wherein the first wireless sensing device is configured to transmit identification information in conjunction with the first parameter data, and wherein detecting the presence of the first wireless sensing device in the predefined area includes receiving the identification information at the detector communicatively connected to the second patient monitor via a host network.

20. The system of claim 13, wherein the second patient monitor is further configured to:
upon receipt of identification information of the first wireless sensing device, display a transfer request on the second display requesting clinician approval to receive data from the first wireless sensing device; and
receive a clinician input approving receipt of first parameter data from the first wireless sensing device prior to operating the second patient monitor to receive the first parameter data.
